(19) United States
(12) Patent Application Publication
(10) Pub. No.: US 2024/0306994 A1
(43) Pub. Date: Sep. 19, 2024

(54) WEARABLE HEALTH MONITORING DEVICE

(71) Applicant: KNOW LABS, INC., Seattle, WA (US)

(72) Inventor: JOHN CRONIN, Seattle, WA (US)

(21) Appl. No.: 18/608,492

(22) Filed: Mar. 18, 2024

Related U.S. Application Data
(60) Provisional application No. 63/490,636, filed on Mar. 16, 2023.

(51) Int. Cl. A61B 5/00; A61B 5/0205; A61B 5/05; A61B 5/11; A61B 5/332; G04G 21/02; G04G 21/04

(52) U.S. Cl. CPC A61B 5/681; A61B 5/02055; A61B 5/7203; G04G 21/025; G04G 21/04; A61B 5/05; A61B 5/1114; A61B 5/332; A61B 5/4857; A61B 5/489; A61B 2560/0443; A61B 2560/0475; A61B 2562/046

(57) ABSTRACT

A wearable health monitoring device comprising a housing and an attachment device detachably coupled to the housing. The attachment device having one or more transmit and receive antennas configured to transmit radio waves and receive a responded portion of the transmitted radio waves over a three-dimensional (3D) space below the skin surface of a user. A processing unit is configured to isolate a signal from a particular location in the 3D space in response to the received radio waves on the one or more receive antennas and output a signal corresponding to the user's health parameters in response to the isolated signal.

### CROSS-REFERENCE TO RELATED APPLICATIONS

This application claims priority to U.S. Provisional Application No. 63/490,636, filed on Mar. 16, 2023, the disclosure of which is incorporated herein by reference in its entirety.

### FIELD OF THE DISCLOSURE

The present disclosure is generally related to a monitoring device and, more particularly, to a wearable apparatus for real-time monitoring of health parameters using RF-range radio frequency signals.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Diabetes is becoming a very common problem worldwide. Diabetes is a chronic disease that affects how the human body treats glucose inside the blood. In a diabetic condition, the pancreas cannot produce insulin, allowing body cells to use glucose. Diabetes can result in severe medical complications, including cardiovascular disease, kidney disease, stroke, foot ulcers, and eye damage if left untreated. Typically, Diabetes is caused by either insufficient insulin production by the pancreas, referred to as “Type 1 diabetes,” or improper insulin response by the body's cells, referred to as “Type 2 diabetes.” Therefore, patients must follow up with doctors regularly to monitor their glucose levels.

Typically, various methods are used by doctors to measure the glucose level inside the blood of the patient. For instance, blood samples are taken by doctors and sent to the labs. However, it takes many days for the doctors to give an accurate result, and in most cases, the patient is required to physically collect the report. Further, a lancet is used to take the patient's blood sample. The lancet is a sharp needle-like object that penetrates through the skin of a finger to draw a blood sample. This is a basic procedure that can be done by the patient easily. However, it is an invasive technique that requires pricking a small hole in the finger, which is not ideal for the patient. Further, it creates chances of spreading pathogens and viruses that may intrude inside the patient's body and infect the blood. Therefore, few health monitoring devices are used in hospitals and clinics that do not require invasive techniques to determine the patient's glucose level. However, these techniques somehow discourage the patients from taking regular health checkups since the patient is required to frequently visit clinics and hospitals. In the prior art, a health monitoring device is incorporated into a watch band.

In order to overcome these drawbacks, many wearable smart devices are used. Such devices are integrated with sensors capable of monitoring patients' health parameters such as blood oxygen percentage, blood pressure, sleeping hours, etc.

Therefore, there is a need to develop a portable and easy-to-use device capable of accurately measuring a user's health parameters. What would be beneficial is a health monitoring device that “attaches to” a watch band and is not integrated into a watch band. That has the benefit of being quickly detached so that the smartwatch can still be used (we don't have to detach watch bands). What is also beneficial is that a health monitoring device to use more area (to have an antenna array) so that all of the components of the health monitoring device do not have to fit everything inside a watch band, so aesthetically, a health monitoring device is used when it's needed versus always on. Our invention can create any form factor independent of the watch band.

### SUMMARY

A wearable health monitoring device. An example off a wearable heath monitoring device includes a housing, an attachment device and a processing unit. The attachment device is detachably coupled to the housing, wherein the attachment device has one or more transmit antennas and one or more receive antennas configured to transmit radio waves and receive responded portion of the transmitted radio waves over a three-dimensional (3D) space below the skin surface of a user. The processing unit is configured to isolate a signal from a particular location in the 3D space in response to the received radio waves on the one or more receive antennas and output a signal that corresponds to health parameters of the user in response to the isolated signal.

In one example, a wearable health monitoring device comprising a flexible strap band that includes a flexible strap portion. The flexible strap band configured for wear over the wrist of a user and detachable coupling to a smartwatch. The flexible strap band including a plurality of antenna bands integrated within the flexible strap portion, the plurality of antenna bands fabricated with one or more transmit antenna bands and one or more receive antenna bands configured to transmit radio frequency (RF) detection signals into the skin of the user and a receive antenna band configured to detect RF return signals resulting from transmitting the RF detection signals into the person, the plurality of antenna bands disposed in an array. The flexible strap band including an electronic device, connected to the plurality of antenna bands and controlling the plurality of antenna bands. The electronic device including a processing unit configured to extract information related to the health parameters of the user for in response to the RF return signals detected from the receive antenna bands and to output a signal that corresponds to health parameters of the user in response to the received RF return signals. The electronic device including a communication module to send the health parameters to the smartwatch. The flexible strap band including a right connector and a left connector at the respective sides of the flexible strap portion, configured to couple magnetically, mechanically, or using hook and loop fasteners to form a loop.

In another example, a wearable health monitoring device comprising a housing and an attachment device detachably coupled to the housing. The attachment device having one or more transmit and receive antennas configured to transmit radio waves and receive a responded portion of the transmitted radio waves over a three-dimensional (3D) space below the skin surface of a user. A processing unit is configured to isolate a signal from a particular location in the 3D space in response to the received radio waves on the one or more receive antennas and output a signal corresponding to the user's health parameters in response to the isolated signal

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a perspective view of a known wearable health monitoring device coupled with a smartwatch;

FIG. 2A illustrates a cross-sectional view of a housing of a wearable health monitoring device coupled to a strap connector according to an embodiment;

FIG. 2B illustrates an isometric view of the housing coupled to the strap connector, according to the embodiment of FIG. 2A;

FIG. 3A illustrates a cross-sectional view of a flexible strap band, according to another embodiment;

FIG. 3B illustrates a cross-sectional view of the flexible strap band of FIG. 3A attached to a magnetic coupler, according to the embodiment;

FIG. 3C illustrates an antenna band array, according to the embodiment;

FIG. 3D illustrates another cross-sectional view of the flexible strap band coupled to a watch display, according to the embodiment;

FIG. 4A illustrates a top view of the flexible strap band coupled with a housing, according to an alternate embodiment;

FIG. 4B illustrates a cross-sectional view of the flexible strap band of FIG. 4A coupled with the housing, according to the alternate embodiment; and

FIG. 4C illustrates an antenna band array, according to the alternate embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

A “Module” is a software or hardware or hybrid set of components that perform an operation of the invention.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

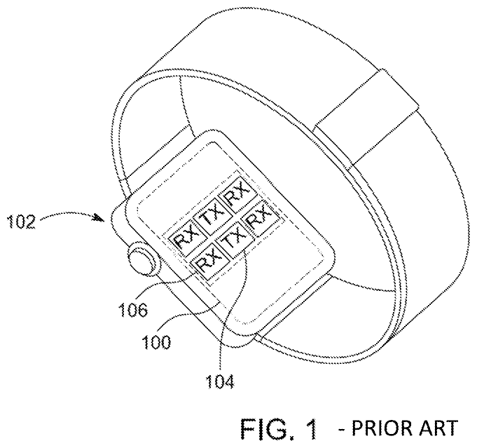

FIG. 1 illustrates a perspective view of a known wearable health monitoring device 100 from the prior art coupled with a smartwatch 102, according to an embodiment. Further information on the construction and operation of the wearable health monitoring device 100 and the smartwatch 102 can be found in US 2020/0187792, the entire contents of which are incorporated herein by reference.

The wearable health monitoring device 100 may be configured to be worn by a user to calculate the user's glucose level and other health parameters. In one embodiment, the wearable health monitoring device 100 and the smartwatch 102 may be specifically placed over a target vein to measure the glucose level.

In the prior art, the wearable health monitoring device 100 may be coupled to other sensing devices, such as, but not limited to, a smartphone, health and fitness bands, protective covers of the smartphones, and smartwatches that may or may not be wearable by the user.

In the prior art, the wearable health monitoring device 100 may be constructed specifically based on the average wrist anatomy of the user. It can be noted that an average depth and/or width of a typical wrist ranges from 4-6 centimeters (cm). It can also be noted that the thickness of the skin on the wrist and elsewhere ranges from 0.60 mm to 3.20 mm. Also, the thickness of subcutaneous tissue ranges from 1.65 mm to 18.20 mm. Therefore, the wearable health monitoring device 100 may transmit radio waves of a specified frequency range to penetrate the dermis and subcutaneous tissue since cephalic and basilica veins are located within these tissue layers.

In the prior art, the wearable health monitoring device 100 may comprise a radio frequency sensing module (not shown), a memory unit (not shown), a communication module (not shown), and a battery (not shown). Further, the radio frequency sensing module may comprise at least one transmitting antenna (TX antenna) 104, at least one receiving antenna (RX antenna) 106, a semiconductor chip (not shown), and a processing unit (not shown).

In the prior art, the semiconductor chip may be configured to generate the radio frequency signals in a frequency range of 120-128 GHz that may have a two-dimensional illumination pattern. It may be noted that the illumination pattern covers a three-dimensional (3D) space or volume underneath the user's skin. Alternatively, the semiconductor chip may comprise a phase-locked loop (PLL) (not shown), a band pass filter (BPF) (not shown), and a frequency mixer (not shown) fabricated over the semiconductor chip. For instance, the semiconductor chip generates an analog signal at a frequency range of 9-11 MHz, and this 9-11 MHz is fed to the PLL, which generates an analog signal in the range of 3-5 GHz frequency. Further, the 3-5 GHz signal is provided to the BPF, which filters the analog signal and passes a signal in the 3-5 GHz range to the mixer.

In the prior art, the semiconductor chip may comprise a frequency synthesizer (not shown) and a frequency multiplier (not shown). The frequency synthesizer may be configured to generate a variety of output frequencies as multiples of a single frequency. Further, the frequency multiplier is configured to generate an output frequency that is an odd or even multiple of its input frequency. Further, the semiconductor chip may comprise the frequency mixer configured to create new frequencies with respect to the applied frequencies targeted on it.

In the prior art, for example, the frequency synthesizer uses the 9-11 MHz signal to produce a 16 GHz signal. The 16 GHz signal is fed to the frequency multiplier to generate a signal at 120 GHz by doubling the frequency. Further, the produced 3-5 GHz signals and the 120 GHz signals are mixed to generate a signal at 122-120 GHz depending on the frequency between the 3-5 GHz signal. Further, the signals are amplified in the range of 120-128 GHZ, and these signals are transmitted under the user's skin surface by at least one TX antenna 104.

In the prior art, the RF signals are responded from the user and received by the at least one RX antenna 106 in the form of electromagnetic energy, which may be further converted into electrical signals. For example, the electromagnetic energy in the 120-128 GHz frequency band is received by at least one RX antenna 106 and converted to a 120-128 GHz electrical signal. The semiconductor chip is configured to amplify the 120-128 GHz electrical signal into the 122-128 GHz frequency range to an amplified output signal.

In the prior art, the amplified 120-128 GHz signal is mixed with the 128 GHz signal from the frequency multiplier with the received 120-128 GHz signal to generate a 3-5 GHz signal corresponding to the electromagnetic energy received by the at least one RX antenna 106. The 3-5 GHz signal is mixed with the 3-5 GHz+2.5 MHz signal to generate a 2.5 MHz signal corresponding to the electromagnetic energy received by the at least one RX antenna 106. Further, the semiconductor chip uses the 2 GHz signal and mixes with the 2 GHz+2.5 MHz signals to generate a 2.5 MHz signal. Successively, the 2.5 MHz signal that corresponds to the electromagnetic energy is converted from an analog signal to a digital signal.

In the prior art, the digital signal is received by the processing unit that is communicatively coupled to the semiconductor chip. Example implementations of the processing unit may include an X86-based processor, a Reduced Instruction Set Computing (RISC) processor, an Application-Specific Integrated Circuit (ASIC) processor, a Complex Instruction Set Computing (CISC) processor, and/or other processors. The processing unit is configured to convert the digital signal into digital data encoded with the health parameter information of the user. In an embodiment, the processing unit first filters the 2.5 MHz signal to remove the negative frequency spectrum and noise outside the desired bandwidth and converts the 2.5 MHz signal to digital data. Further, the semiconductor chip may comprise a decimation filter (not shown) configured to reduce the sampling frequency of the received signal. It can be noted that the decimation filter may be integrated with an analog-to-digital converter (ADC) within the semiconductor chip. The processing unit further decimated the digital data sampled at 9-11 MHz. It may be noted that the digital data is decimated to reduce the amount of data by selectively discarding a portion of sampled data to uncover meaningful information from the digital data.

In the prior art, the output of the decimation filter is the digital data that is representative of the electromagnetic energy received at the corresponding receiving antenna. Alternatively, signal processing techniques may be used to achieve beamforming, Doppler effect processing, and/or leakage mitigation in order to separate a desired signal from other undesirable signals. The digital signal processing of incoming signals may use Kalman filters to smooth out noisy data. In one aspect, the digital signal processing of received signals may include digitally merging receive chains. In another aspect, multiple digital signal processing techniques may be utilized to achieve beamforming, Doppler effect processing, and range. Digital signal processing can be accomplished in a digital signal processor (DSP).

In the prior art, RF-range radio frequency signals may be delivered beneath the skin to highlight anatomical characteristics. Blood flowing via veins such as the basilic and cephalic veins moves relative to other anatomical characteristics in the region surrounding the wrist. Thus, the processing unit employs the Doppler effect theory and associated signal processing to filter for signals corresponding to blood flowing inside veins relative to other signals corresponding to stationary objects. It can be noted that the stationary objects may correspond to bone and fibrous tissue such as muscle and tendons. The signals that correspond to the flowing blood may be recognized and isolated. The isolated signals may then be utilized to calculate a health metric, such as blood glucose levels.

In the prior art, the Doppler effect theory may be applied for processing received signals to separate the signals corresponding to the flowing blood moving relative to the transmitting and receiving antennas from the signals corresponding to stationary objects. Although the approaches discussed above focus on monitoring the blood glucose level, the disclosed techniques may also be relevant to monitor other health metrics such as blood pressure and heart rate.

In the prior art, the digital data processed by the processing unit is further saved within the memory unit. The memory unit may include suitable logic, circuitry, and/or interfaces that may be configured to store a machine code and/or a computer program with at least one code section executable by the processor unit. Examples of implementation of the memory may include, but are not limited to, Random Access Memory (RAM), Read Only Memory (ROM), Hard Disk Drive (HDD), and/or a Secure Digital (SD) card. In an embodiment, the digital information processed by the processing unit is saved within the memory unit in the form of a database.

In the prior art, the database may be segregated into a raw data sample and derived data fetched from the user. In one embodiment, the raw data sample may correspond to standard data or a threshold limit for performing a specific health parameter. In one embodiment, the derived data may correspond to data calculated or evaluated from a sample. The raw data sample may be used as a training data set as well as a reference point for the wearable health monitoring device 100 to determine the accurate health results of the user. It may be noted that the raw data sample may include data sets of different parameters to allow the wearable health monitoring device 100 to fetch the derived data at different conditions. In one embodiment, the raw data samples include data sets specific to gender, age, specific to weather conditions, specific to regions, etc.

In the prior art, the wearable health monitoring device 100 may also be integrated with the communication module. The communication module may be configured to transfer the derived data stored within the database to the smartwatch 102 for the user. Further, the communication module may provide a medium through which the wearable health monitoring device 100 may communicate with a cloud network in the network environment or with each other. Such communication may facilitate the user to securely save the data in a cloud database and enable access to the derived data from multiple locations and devices.

In the prior art, examples of the communication network may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). Various devices may be configured to connect the communication network to the cloud via various wired and wireless communication protocols. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In an embodiment, the wearable health monitoring device 100 may also be integrated with the battery that may be configured to power all the elements of the wearable health monitoring device 100.

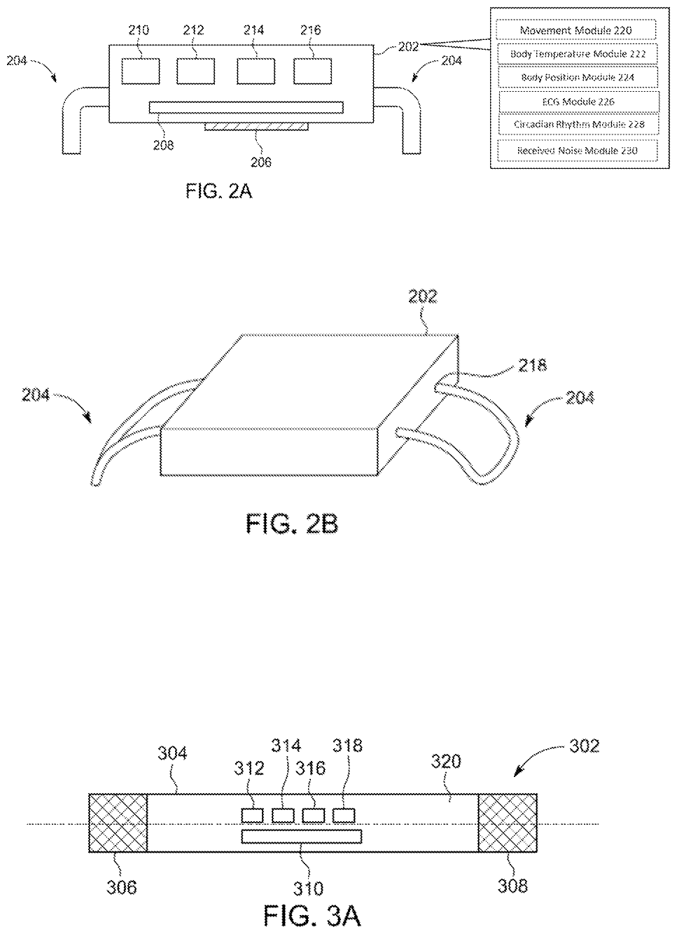

FIG. 2A illustrates a cross-sectional view of a housing 202, which connects to a smartwatch using connectors 204

The housing 202 may be coupled to a smart device such as a smartwatch or smart band configured to measure the user's glucose level. The housing 202 may include an antenna band 206, a connector 208, a memory unit 210, a communication module 212, a processing unit 214, a battery 216, and a strap insertion point 218.

The antenna band 206 may have one or more TX and RX antenna bands (not shown) that may be configured to transmit radio waves into the user's skin and receive the responded radio waves from blood. In one embodiment, the antenna band 206 may be positioned underneath the housing 202, as shown in FIG. 2A, such that once the housing 202 is placed over the wrist of the user, the antenna band 206 comes in proximity to the user's skin.

In one embodiment, the antenna band 206 may have a suitable dimension to accommodate the housing 202. It can be noted that one or more TX and RX antenna bands may have a length of 1.2 millimeters (mm) to 1.3 mm and a width of 1.1 mm to 1.25 mm. Such configuration of the antenna band 206 may enable one or more TX and RX antenna bands to produce radio waves in a range of 500 MHZ to 300 GHZ GHz to penetrate the user's skin.

In one embodiment, the one or more TX and RX antenna bands of the antenna band 206 may be fabricated over a single semiconductor substrate. In one exemplary embodiment, the semiconductor substrate may have dimensions of approximately 4 mm to 6 mm by 4 mm to 6 mm. It can be noted that the semiconductor substrate may be referred to as the footprint of the semiconductor chip. In one embodiment, the semiconductor chip's size and the antenna band 206 may differ based on their application and/or placement.

Further, the antenna band 206 may be coupled to the connector 208. It can be noted that the connector 208 is an electromechanical component configured to establish an electrical contact between the antenna band 206 and other elements of the housing 202. In one embodiment, the connector 208 may be configured with a male component, a plug, a female component, or a socket. It can be noted that electrical circuits or wires coming from the antenna band 206 and other elements of the housing 202 may interact at the established electrical contact of the connector 208.

Further, the processing unit 214 is configured to process the signals in response to the responded radio waves received from the at least one RX antenna 106. The radio waves are responses from multiple wanted objects such as blood, and unwanted objects, such as bones, ligaments, skin, etc. Therefore, the processing unit 214 may be configured to isolate a signal from a particular location in the 3D space, to locate the veins in response to the radio waves received by the one or more RX antenna bands. Further, the processing unit 214 may output a signal corresponding to the user's health parameter in response to the isolated signal.

Further, the processing unit 214 may be communicatively coupled to the memory unit 210. The memory unit 210 may be configured to store a set of instructions executed by one or more processors of the processing unit 214 to determine the required results. In one embodiment, the memory unit 210 may also be configured to store the data related to the health parameters processed by the processing unit 214. Examples of the memory unit 210 may include, but are not limited to, Random Access Memory (RAM), Read Only Memory (ROM), Hard Disk Drive (HDD), and/or a Secure Digital (SD) card.

Further, the strap connector 204 may comprise left and right strap connectors configured to couple the housing 202 to a smartwatch. The strap connector 204 may be attached to the housing 202 through the strap insertion point 218. The strap connector 204 may be configured to detachably attach the housing 202 with a strap (not shown) of the smartwatch worn by the user. This may allow the user to wear the housing 204 over the wrist area to monitor and communicate data to a smartwatch to display health parameters.

The housing 202 may include a movement module 220 that includes at least one sensor from the group of an accelerometer, a gyroscope, an inertial movement sensor, or other similar sensor. The movement module 220 may have its own processor or utilize the processing unit 214 to calculate the user's movement. Motion from the user will change the blood volume in a given portion of their body and the blood flow rate in their circulatory system. This may cause noise, artifacts, or other errors in the real-time signals received by the antenna band 206. The movement module 220 may compare the calculated motion to a motion threshold stored in the memory unit 210. For example, the motion threshold could be movement of more than two centimeters in one second. The motion threshold could be near zero to ensure the user is stationary when measuring to ensure the least noise in the RF signal data. When calculated motion levels exceed the threshold, the motion module may flag the RF signals collected at the time stamp corresponding to the motion as potentially being inaccurate. In some embodiments, the movement module 220 may compare RF signal data to motion data over time to improve the accuracy of the movement threshold. The movement module 220 may alert the user, such as with an audible beep, warning, text message, or alert to a connected mobile device. The alert would signal the user that they are moving too much to get an accurate measurement.

The housing 202 may include a body temperature module 222 that includes at least one sensor from the group of a thermometer, a platinum resistance thermometer (PRT), a thermistor, a thermocouple, or another temperature sensor. The body temperature module 222 may have its own processor or utilize the processing unit 214 to calculate the user's temperature or the user's environment. The user's body temperature, the environmental temperature, and the difference between the two will change the blood volume in a given part of their body and the blood flow rate in their circulatory system. Variations in temperature from normal body temperature or room temperature may cause noise, artifacts, or other errors in the real-time signals received by the antenna band 206. The body temperature module 222 may compare the measured temperature to a threshold temperature stored in the memory unit 210. For example, the environmental temperature threshold may be set at zero degrees Celsius because low temperatures can cause a temporary narrowing of blood vessels which may increase the user's blood pressure. When the measured temperature exceeds the threshold, the body temperature module 222 may flag the RF signals collected at the time stamp corresponding to the temperature as potentially inaccurate. In some embodiments, the body temperature module 222 may compare RF signal data to temperature data over time to improve the accuracy of the temperature threshold. The body temperature module 222 may alert the user, such as with an audible beep, warning, text message, or alert to a connected mobile device. The alert would signal to the user that their body or environmental temperature is not conducive to getting an accurate measurement.

The housing 202 may include a body position module 224 that includes at least one sensor from the group of an accelerometer, a gyroscope, an inertial movement sensor, or other similar sensor. The body position module 224 may have its own processor or utilize the processing unit 214 to estimate the user's position. The user's body position may change the blood volume in a given part of their body and the blood flow rate in their circulatory system. This may cause noise, artifacts, or other errors in the real-time signals received by the antenna band 206. The body position module 224 may compare the estimated position to a body position threshold stored in the memory unit 210. For example, the smartwatch 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 224 may flag the RF signals collected at the time stamp corresponding to the body position as potentially being inaccurate. In some embodiments, the body position module 224 may compare RF signal data to motion data over time to improve the accuracy of the body position threshold. The body position data may also be used to estimate variations in parameters such as blood pressure that corresponds to the body position data so as to improve the accuracy of the measurements taken when the user is in that position. The body position module 224 may alert the user, such as with an audible beep, warning, text message, or alert to a connected mobile device. The alert would signal to the user that their body position is not conducive to getting an accurate measurement.

The housing 202 may include an ECG module 226 that includes at least one electrocardiogram sensor. The ECG module 226 may have its own processor or utilize the processing unit 214 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the antenna band 206. The ECG module 226 may compare the measured cardiac data to a threshold stored in the memory unit 210. For example, the threshold may be a pulse above 160 bpm, as the increased blood flow volume may cause too much noise in the received RF signal data to accurately measure the blood glucose. When the ECG data exceeds the threshold, the ECG module 226 may flag the RF signals collected at the time stamp corresponding to the ECG data as potentially being inaccurate. In some embodiments, the ECG module 226 may compare RF signal data to ECG data over time to improve the accuracy of the ECG data threshold or to improve the measurement of glucose at a given point in the cycle between peak and minimum cardiac output. The ECG module 226 may alert the user, such as with an audible beep, warning, text message, or alert to a connected mobile device. The alert would signal to the user that their heart rate is not conducive to getting an accurate measurement or requires additional medical intervention.

The housing 202 may include a circadian rhythm module 228 that includes at least one sensor measuring actigraphy, wrist temperature, light exposure, and heart rate. The circadian rhythm module 228 may have its own processor or utilize the processing unit 214 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the antenna band 206. The circadian rhythm module 228 may compare the circadian data to a threshold stored in the memory unit 210. For example, the threshold may be set as less than 6 hours of sleep in the last 24 hours. When the observed circadian health data exceeds the threshold, the circadian rhythm module 228 may flag the RF signals collected at the time stamp corresponding to circadian health as potentially being inaccurate or needing an adjustment to account for the expected increase in the user's blood pressure. In some embodiments, the circadian rhythm module 228 may compare RF signal data to sleep data over time to improve the accuracy of the circadian rhythm thresholds. The circadian rhythm module 228 may alert the user, such as with an audible beep, warning, text message, or alert to a connected mobile device. The alert would signal to the user that their recent sleep patterns are not conducive to getting an accurate measurement.

The housing 202 may include a received noise module 230 that includes at least one sensor measuring background signals such as RF signals, Wi-Fi, and other electromagnetic signals that could interfere with the signals received by the antenna band 206. The received noise module 230 may have its own processor or utilize the processing unit 214 to calculate the level of background noise received. Background noise may interfere with or cause noise, artifacts, or other errors or inaccuracies in the real-time signals received by the antenna band 206. The received noise module 230 may compare the level and type of background noise to a threshold stored in the memory unit 210. The threshold may be in terms of field strength (volts per meter and ampere per meter) or power density (watts per square meter). For example, the threshold may be RF radiation at greater than 300 μW/m2. When the background noise data exceeds the threshold, the received noise module 230 may flag the RF signals collected at the time stamp corresponding to background noise levels as potentially being inaccurate. In some embodiments, the received noise module 230 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement.

In one embodiment, the strap connector 204 may be structured in a loop, as shown in FIG. 2B. Such configuration of the strap connector 204 may enable to flexibly wrap of the strap of the smartwatch 102 around the loop. In another embodiment, the strap connector 204 may be integrated with a locking pin unit (not shown). The locking pin unit may have a reciprocating pin (not shown) housed inside the strap connector 204. To attach the strap of the smartwatch 102 with the strap connector 204, the user may push the reciprocating pin inside the strap connector 204 and simultaneously position the strap along the strap connector 204. Once the strap is positioned, the reciprocating pin may be released and inserted inside one end of the strap, thereby making an interlocking orientation to lock the strap with the housing 202.

In one embodiment, the strap connector 204 may facilitate the connection of multiple types of straps to the housing 202. Such versatile application of the strap connector 204 may allow the user to couple the housing 202 with multiple types of straps such as bracelets, bands, customized accessories, etc.

For example, Alex uses the wearable health monitoring device 100 to check their glucose level at 10:00 AM. Firstly, Alex attaches each side of a stretchable wristband with the strap connectors 204. The wristband may be a rigid piece. The wristband may extend partially around the wrist.

Further, the antenna band 206 is activated to transmit and receive the radio waves of frequency range 120-128 GHz. The processing unit 214 receives a responded portion of the radio waves and determines the glucose level of Alex displayed over a display screen of the housing 202. The display screen shows the blood glucose level of Alex as 110 mg/dL. Other health parameters are shown to Alex, indicating he has a heart rate of 74 BPM, Blood Pressure of 111/70, and SpO2 of 96.5%.

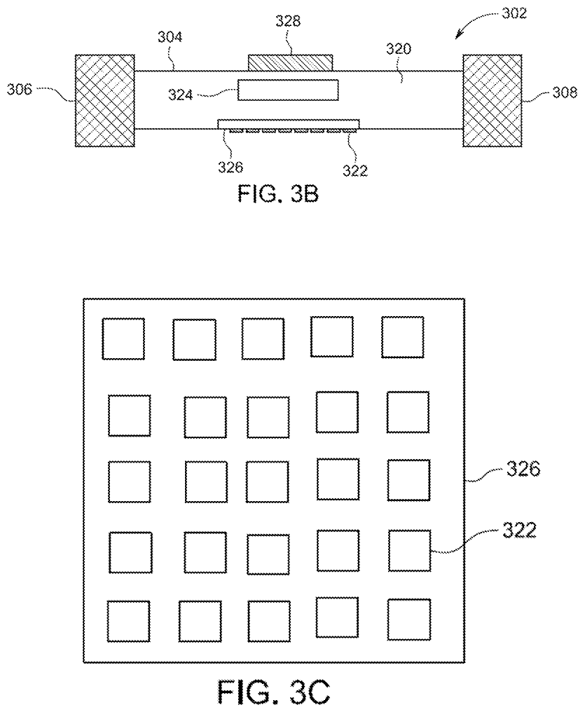

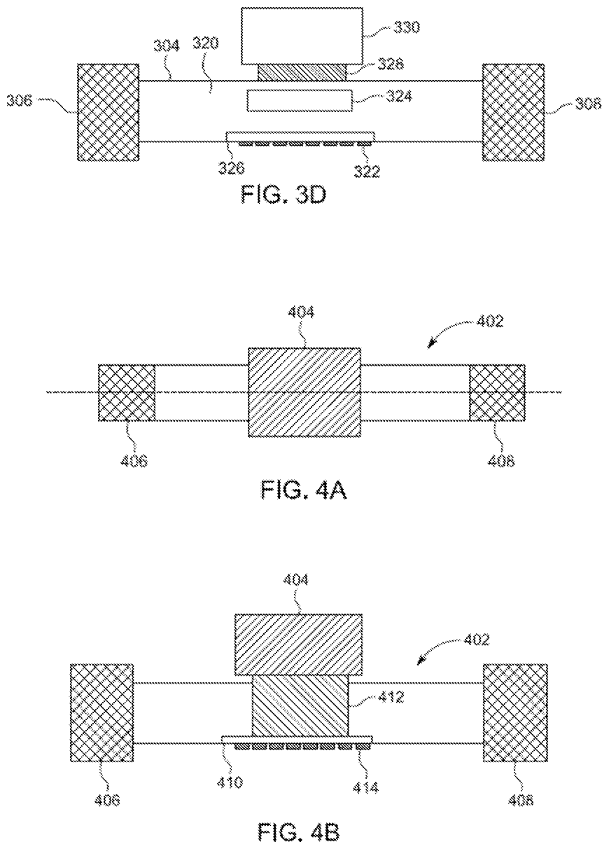

FIG. 3A illustrates a cross-sectional view of a flexible strap band 302, according to an embodiment. FIG. 3A is described in conjunction with FIGS. 3B-3D.

The flexible strap band 302 may be worn by the user to measure the glucose level. The flexible strap band 302 may be configured in a manner to measure the glucose level and display over the smartwatch 102 connected to the flexible strap band 302 via a connecting means (not shown). The flexible strap band 302 may comprise a flexible strap 304, a left buckle 306, a right buckle 308, a connector 310, a memory unit 312, a processing unit 314, a communication module 316, a battery 318, and a covering 320 and an antenna band 322.

In one embodiment, the flexible strap 304 may be configured to be worn over the wrist of the user. The flexible strap 304 may be made from a flexible material selected from a group of materials such as but not limited to, rubber, polyester, leather, metal, steel, and polyethylene (PE). It can be noted that the flexible strap 304 may be worn over the wrist of different sizes and orientations. Further, the flexible material may allow the flexible strap 304 to be placed tightly over the skin surface of the user such that the glucose level and other health parameters are measured with high accuracy. In one embodiment, each side of the flexible strap 304 may be attached with the left buckle 306 and the right buckle 308. The flexible strap may be attached to a portion of the smartwatch wristband.

The left buckle 306 and the right buckle 308 may be coupled to each other to form a loop either magnetically or mechanically or using hook and loop fasteners (not shown). The left buckle 306 and the right buckle 308 may be integrated with a magnet (not shown) with opposite poles to couple the right buckle 308 with the left buckle 306. Further, the left buckle 306 and the right buckle 308 may be attached mechanically using loop and pin units (not shown). In one embodiment, the left buckle 306 may be attached with a loop, and the right buckle 308 may be provided with a pin such that the pin may be passed through the loop and inserted via one or more holes carved over the surface of the flexible strap 304. In another embodiment, each end of the left buckle 306 and the right buckle 308 may be fabricated with the hook and loop fasteners.

Further, the flexible strap 304 may be integrated with the antenna band 322, as shown in FIGS. 3B and 3C. As discussed above, the antenna band 322 is an antenna band fabricated with one or more TX antenna bands and one or more RX antenna bands configured to transmit and receive radio waves. The transmitted radio waves penetrate the user's skin and respond from the target vein. Further, the one or more RX antenna bands intercept the responded radio waves. Further, the antenna band 322 is coupled to an electronic device 324 having the connector 310, the memory unit 312, the processing unit 314, the communication module 316, and the battery 318. Further, the received radio waves are processed by the processing unit 314. The processing unit 314 is configured to extract information related to the user's health parameters from the RX antennas via the connectors 310 and store the data in memory 312 to later analyze the data and send the results through the communication module 316 to a smartwatch.

In one embodiment, the processing unit 314 and other electronic components of the flexible strap 304 may be communicatively coupled to the antenna band 322 through the connector 310. It can be noted that the connector 310 may be an electromechanical unit that provides a junction to connect one or more electronic units with each other. The connector 310 may comprise one or more ports (not shown) connected with the antenna band 322 and other electronic components to transmit the electrical signals. Further, the processing unit 314 may be communicatively coupled to the memory unit 312. In one embodiment, the memory unit 312 may be configured to save the data fetched by the processing unit 314. The data may include the user's health parameters measured by the antenna band 322.

Further, the antenna band 322 may be fabricated over an antenna array 326, as shown in FIG. 3B. In one embodiment, the antenna array 326 may be the single semiconductor substrate. In one exemplary embodiment, the semiconductor substrate may have dimensions of approximately 3 mm to 5 mm by 3 mm to 5 mm. It can be noted that the semiconductor substrate may be referred to as the footprint of the semiconductor chip. The antenna band 322 may have multiple transmit and receive antennas configured to transmit and receive radio signals of multiple frequency ranges. In one exemplary embodiment, the transmit and receive antennas of the antenna band 322 may be embedded within the antenna array 326 for the smooth texture of the flexible strap band 302.

Further, the flexible strap 304 may be attached to a magnetic coupler 328. The magnetic coupler 328 may be configured to securely attach the flexible strap 304 with a watch display 330, as shown in FIG. 3D. In one embodiment, the magnetic coupler 328 may be attached to a bottom surface (not shown) of the watch display 330. In one embodiment, the magnetic coupler 328 may also be configured to be coupled with a smartphone, a smartphone case, a fitness band display, etc. Such configuration of the magnetic coupler 328 may allow to display of the derived health parameter of the user by using the flexible strap 304 and display over the connected watch display 330.

Further, the flexible strap 304 may comprise the communication module 316. The communication module 316 may transmit the information from the memory unit 312 to the watch display 330. The user's glucose level may be displayed over the watch display 330. Further, the flexible strap 304 may be layered with the covering 320. The covering 320 may be used to securely pack the electrical components inside the flexible strap 304. In one embodiment, the battery 318 may be integrated inside the flexible strap band 302 to power the electrical components.

For example, Sam wears the flexible strap band 302 over his left-hand wrist. The antenna band 322 is placed in contact with the skin of Sam, and the magnetic coupler 328 is at the top side of the flexible strap band 302 to attach a Google watch screen with the magnetic coupler 328. Upon activating the flexible strap 304, the health parameters of Sam are determined and sent to the Google watch screen via the communication module 316. The Google watch screen shows the health parameters in a tabular form shown as:

| | | |
|---|---|---|
| Blood Glucose level (mg/dL) | 138 | 11:00 AM |
| Heart rate (BPM) | 90 | 11:00 AM |
| Blood Pressure (mmHg) | 128/70 | 11:00-11:01 AM |

In one embodiment, the antenna band 322 may be configured to cover the maximum area over the surface of the wrist of the user with the multiple transmit and receive antennas of the antenna band 322, as shown in FIG. 3C. In one embodiment, the processing unit 314 may be configured with a method to sequentially activate and deactivate each of the multiple transmit and receive antennas. The activation and deactivation of the multiple transmit and receive antennas may allow for picking up radio signals from at least one target vein.

In an alternative embodiment, the processing unit 314 may be configured to activate and/or deactivate the multiple transmit and receive antennas of the antenna band 322. Such activation and deactivation of each of the multiple transmit and receive antennas may enable the antenna band 322 to receive better-responded radio signals from the at least one target vein. For example, Sam wears the flexible strap band 302 over his left wrist, and one transmit and receive antenna of the antenna band 322 are activated simultaneously to transmit and receive radio frequency signals of range 120-128 MHz underneath the wrist.

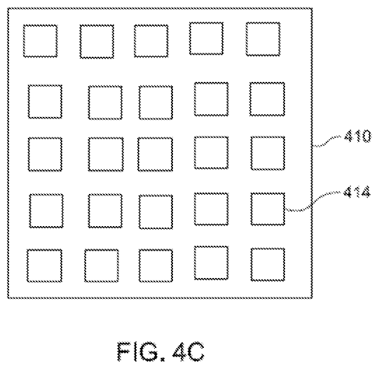

FIG. 4A illustrates a top view of a flexible strap band 402 coupled with a housing 404, according to an embodiment. FIG. 4A will be described in conjunction with FIG. 4B.

The flexible strap band 402 may comprise a left buckle 406, a right buckle 408, an antenna array 410, and a connector 412. The left buckle 406 and the right buckle 408 may be coupled to each other using the hook and loop fasteners, the loop and pin units, etc.

Further, the antenna array 410 may be integrated into the flexible strap band 402. The antenna array 410 may comprise one or more antenna bands 414. The one or more antenna bands 414 may be sequentially and/or randomly activated and deactivated to cover a wider area of the skin surface of the user to extract the most accurate results. The antenna array 410 may be arranged with the flexible strap band 402 so that when the flexible strap band 402 is worn over the wrist of the user, the antenna array 410 contacts the user's skin to measure the health parameters accurately.

Further, the connector 412 may be attached from one end to the antenna array 410. In one embodiment, the connector 412 may be an electromechanical component configured to detachably couple the antenna array 410 to the housing 404. In one embodiment, the housing 404 may comprise a processing unit (not shown), a memory unit (not shown), a communication module (not shown), and a battery (not shown). It can be noted that the connector 412 provides a channel to allow the flow of electrical signals communicated through the antenna array 410 to the processing unit. The processing unit extracts the user's health parameters from the processed received signals that are further saved to the memory unit. In one embodiment, the housing 404 may also be integrated with the communication module configured to establish wireless communication with other electronic devices to allow the user to access or share the output information in the form of health parameters to other devices.

For example, Murphy wears the flexible strap band 402 over his left-hand wrist. The flexible strap band 402 is worn by Murphy such that the antenna array 410 gets placed in contact with the skin. Further, Murphy attaches a smart display screen with the connector 412. Upon activating the flexible strap band 402, the health parameters of Murphy are determined and sent to the smart display screen via the communication module. The smart display screen shows a Heart rate of 76 BPM, Blood Pressure of 110/72, and SpO2 of 98%.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as disclosed above.

What is claimed is:

1. A wearable health monitoring device, comprising:
a flexible strap band including a flexible strap portion, configured for wear over the wrist of a user and detachable coupling to a smartwatch, including:
a plurality of antenna bands integrated within the flexible strap portion, the plurality of antenna bands fabricated with one or more transmit antenna bands and one or more receive antenna bands configured to transmit radio frequency (RF) detection signals into the skin of the user and a receive antenna band configured to detect RF return signals resulting from transmitting the RF detection signals into the person, the plurality of antenna bands disposed in an array;
an electronic device, connected to the plurality of antenna bands and controlling the plurality of antenna bands, including:
a processing unit configured to extract information related to the health parameters of the user for in response to the RF return signals detected from the receive antenna bands and to output a signal that corresponds to health parameters of the user in response to the received RF return signals; and
a communication module to send the health parameters to the smartwatch; and
a right connector and a left connector at the respective sides of the flexible strap portion, configured to couple magnetically, mechanically, or using hook and loop fasteners to form a loop.

2. The wearable health monitoring device of claim 1, wherein the electronic device is contained within the flexible strap portion.

3. The wearable health monitoring device of claim 1, wherein the electronic device is contained within a housing independent from the flexible strap portion.

4. The wearable health monitoring device of claim 1, wherein the electronic device further contains a memory unit configured to store data related to health parameters extracted by the processing unit.

5. The wearable health monitoring device of claim 1, wherein the array the plurality of antenna bands are disposed is a single semiconductor substrate.

6. The wearable health monitoring device of claim 1, wherein the electronic device is connected to the plurality of antenna bands by an electromechanical unit providing a junction.

7. The wearable health monitoring device of claim 1, wherein the flexible strap portion includes material selected from one or more of: rubber; polyester, leather, metal, steel, and polyethylene.

8. The wearable health monitoring device of claim 1, wherein the health parameters of the user include at least one of: blood glucose level, heart rate, blood pressure, and oxygen saturation.

9. The wearable health monitoring device of claim 1, further comprising at least one of:
a motion sensor that detects motion of the user during transmission of the RF detection signals by the one or more transmit antenna bands and during detection of the RF signals by the one or more receive antenna; and
a body position sensor that detects the body position of the user during transmission of the RF detection signals by the one or more transmit antenna bands and during detection of the RF signals by the one or more receive antenna bands.

10. The wearable health monitoring device of claim 1, further comprising at least one of:
a body temperature sensor that detects the temperature of the user during transmission of the RF detection signals by the one or more transmit antenna bands and during detection of the RF signals by the one or more receive antenna bands; and
an ECG sensor that senses the heartbeat of the user during transmission of the RF detection signals by the one or more transmit antenna bands and during detection of the RF signals by the one or more receive antenna bands;

11. The wearable health monitoring device of claim 1, further comprising at least one of:
an circadian rhythm sensor that senses circadian data of the user during transmission of the RF detection signals by the one or more transmit antenna bands and during detection of the RF signals by the one or more receive antenna bands; and
an ECG background noise sensor that senses background noise around the user during transmission of the RF detection signals by the one or more transmit antenna bands and during detection of the RF signals by the one or more receive antenna bands.

12. The wearable health monitoring device of claim 1, wherein the processing unit is configured to isolate a signal from a particular location in 3D space to locate veins in response to RF signals received by the receive antenna bands.

13. The wearable health monitoring device of claim 1, wherein the flexible strap portion includes a covering,

14. The wearable health monitoring device of claim 1, wherein the plurality of antenna bands disposed in an array are configured to be sequentially activated and deactivated.

15. The wearable health monitoring device of claim 1, wherein health parameters sent to the smartwatch via the communication module are sent wirelessly.

16. A wearable health monitoring device, comprising:
a housing;
an attachment device detachably coupled to the housing, wherein the attachment device having one or more transmit antennas and one or more receive antennas are configured to transmit radio waves and receive responded portion of the transmitted radio waves over a three-dimensional (3D) space below a skin surface of a user; and
a processing unit configured to isolate a signal from a particular location in the 3D space in response to the received radio waves on the one or more receive antennas and output a signal that corresponds to health parameters of the user in response to the isolated signal.

17. The wearable health monitoring device of claim 16, wherein the electronic device further contains a memory unit configured to store data related to health parameters of the user.

18. The wearable health monitoring device of claim 17, wherein the processing unit is further configured to isolate a signal from a particular location in 3D space to locate veins in response to radio wave signals received by the receive antennas.
